SEQUENCE LISTING (5 sequences)

SEQ ID NO: 1 - protein, 5 aa, Artificial sequence
  Synthetic Peptide
SSSEE

SEQ ID NO: 2 - protein, 25 aa, Artificial sequence
  Synthetic Peptide
RELEELNVPGEIVESLSSSEESITR

SEQ ID NO: 3 - protein, 21 aa, Artificial sequence
  Synthetic Peptide
QMEAESISSSEEIVPDSVEQK

SEQ ID NO: 4 - protein, 21 aa, Artificial sequence
  Synthetic Peptide
KNTMEHVSSSEESIISNETYK

SEQ ID NO: 5 - protein, 25 aa, Artificial sequence
  Synthetic Peptide
NANEEEYSIGSSSEESAEVATEEVK

(19) United States
(12) Patent Application Publication
(10) Pub. No.: US 2009/0305947 A1
(43) Pub. Date: Dec. 10, 2009

(54) LIVESTOCK MANAGEMENT FOR IMPROVED REPRODUCTIVE EFFICIENCY

(75) Inventor: Jose Mario Iscovich, Gan-yavne (IL)

Correspondence Address: FENNEMORE CRAIG, 3003 NORTH CENTRAL AVENUE, SUITE 2600, PHOENIX, AZ, 85012, US

(73) Assignee: Mileutis Ltd

(21) Appl. No.: 12/296,523

(22) PCT Filed: Apr. 11, 2007
(86) PCT No.: PCT/IL2007/000469

§ 371 Date: Mar. 23, 2009

Related U.S. Application Data
(60) Provisional application No. 60/790,784, filed on Apr. 11, 2006.

(51) Int. Cl. A61K 38/17; A61P 15/08

(52) U.S. Cl. 514/7; 514/12

(57) ABSTRACT

The present invention relates to methods for controlling the reproductive cycles and weaning in livestock, particularly to the use of casein-derived peptides for estrus induction and reduction of anestrus intervals, enabling early weaning without negatively affecting the livestock welfare. The present invention further relates to estrus synchronization in a livestock herd and to livestock management programs.

### FIELD OF THE INVENTION

The present invention relates to methods for controlling the reproductive cycles and weaning in livestock, particularly to the use of casein-derived peptides for estrus induction and synchronization leading to an increase in pregnancy rate and for reduction of anestrus intervals, enabling early weaning without negatively affecting the livestock welfare.

### BACKGROUND OF THE INVENTION

Productivity of a livestock herd, particularly meat producing livestock, depends largely on the reproductive efficiency of the herd, and is measured in terms of pregnancy rate, productivity-energy intake ratio, pre- to post-parturition body condition score dynamics, and the weight value of progenies. The critical requirements for any effective estrus cycle regimen are a predictable and high estrus and ovulation responses. The most important parameter to individually evaluate the reproductive efficiency is the interval between sequential parturitions. In economic terms, for example in cows, the period between calving to calving should not exceed the optimum period of one year, i.e. one calving per cow per year. The main determining factor of this interval is the parturition-conception interval. Prolonged postpartum anestrus is a major limitation to high reproductive efficiency. The postpartum interval (PPI) is defined as the interval between parturition and the following estrus. It has been shown that pregnancy percentage falls linearly when the parturition-estrus interval increases from 60 to 120 days. Moreover, weight of the calving livestock and their progeny decreases significantly when the postpartum interval increases.

In year-round calving herds, between 11% and 38% of cows are reported as anestrus at 50 or 60 days after calving. In seasonally calving dairy herds, between 13 and 48% of cows are diagnosed as anovulatory anestrus at the start of the breeding period. Ovulation and estrus after calving are delayed when the positive feedback effects of estradiol on release of luteinizing hormone (LH) from the pituitary, and circulating concentrations of metabolic hormones such as insulin and insulin-like growth factor-I, are reduced by a variety of environmental factors. The main factors are limited energy intake, lower body reserves, increased partitioning of energy to milk production, suckling, and diseases around the time of calving (periparturient diseases).

The interval from calving to first postpartum ovulation is characterized by a period of increasing pulsatile release of LH, associated with the growth and development of ovarian follicles. In order for those follicles to mature and ovulate, gonadotropic support must be sufficient to stimulate increased production of estradiol, which can induce a preovulatory flow of LH and follicle stimulating hormone (FSH). Calf presence is associated with a delay in the onset of first postpartum ovulation in beef cows, and suckling has a suppressive influence on the onset of estrus through a low concentration of LH caused by a low frequency, pulsatile secretion of LH. This suppressive effect is independent of neurosensory pathways within the teat or udder, and maternal-offspring bonding is considered as the essential component of the suckling-induced prolonged PPI in livestock. Removal of the suckling effect results in a rapid increase in LH pulse frequency that is sufficient to stimulate first ovulation, a response not dependent on the level of postpartum nutrition. Other factors that have been identified as influencing the duration of the PPI are nutrition before and after calving, and season and periparturient diseases. The occurrence of clinical diseases such as mastitis, metritis, vaginitis, severe lameness and ketosis during the first month after calving were all reported to be significant risk factors for an extended PPI. In summary, a prolonged PPI is observed when the increase in release of LH and/or metabolic signals is delayed by suckling, in the event of low energy intake and/or low body reserves, when there is an increased partitioning of energy to milk production, or when the livestock welfare is negatively affected due to an increased stress from disease, mammary gland/udder pressure due to weaning, or high environmental temperatures.

The early resumption of estrus cycles following calving is important for high reproductive efficiency in both year-round and seasonally calving herds. For pasture-based production systems, timing of calving is usually set to optimize the use of maximum pasture growth rate in spring and early summer. To maintain an optimal calving distribution during the season, a high conception rate during the early part of the breeding period is an important prerequisite. To maintain a maximum 365-days calving interval, cows need to conceive on average by about eighty days after calving. Delays in the commencement of ovulation and expression of estrus are associated with reduced conception and pregnancy rates and increased intervals from calving to conception.

Treatment options for cows with an extended PPI include hormonal and management strategies. Hormonal treatments that include a period of, for example, progesterone supplementation result in the majority of treated animals displaying estrus with a subsequent luteal phase of normal duration and improved pregnancy rates compared with untreated controls. Hormonal interventions tend to have more predictable outcomes compared with management changes, such as manipulating body condition or dietary intake after calving, and usually have some estrous synchronization effect, thus facilitating the use of artificial insemination. For example, U.S. Pat. No. 6,939,558 discloses intravaginal devices containing progesterone that can be used as an estrus inductor in an organism, such as bovine, swine, equine, and the like.

However, responses to any hormonal treatment are variable and depend on the stage of the follicular wave and the intricacies of control mechanisms that play a role in regulating the sequential progression of follicles. Using hormones for estrus synchronization may result in reduced fertility as well as in secondary effects, thus not achieving the goal of increasing the reproductive efficiency.

Farm animal welfare is of increasing public concern in modern societies in the last decades (Broom D M 1992 In: Phillips et al., Edts. Farm Animals and the Environment CAB Wallingford UK pp 245-253). Recent development in housing and management practices of farm animals under intensive production systems reflects the increase in moral concerns of animal welfare (Fregonesi J A et al. 2001 Livestock Production Sci. 68:205-216; Fregonesi et al. 2002 Livestock Production Sci. 78:245-257). Improvement of animal welfare is defined as the prevention of suffering and increasing the presence of positive feelings, typically called comfort or pleasure (Broom, supra). Measurements of impaired biological functioning, particularly those connected to decreased health and increased physiological stress responses, are used to evaluate the welfare status of farm animals.

In the beef and dairy industries, lactating animals in herds go through controlled cycles of milking and pregnancy, as such regime contributes to a significant increase in calve and milk production. In current management of dairy herds, for example cows and goats, there is a significant overlap between lactation and pregnancy, wherein a “dry period” is imposed between 50 to 70 days prior to parturition by cessation of milking. This regime is set to compromise between the need to induce involution, a necessary process for subsequent healthy lactating period, and the requirement for high milk production all year long. In line with the management principle of controlling reproduction efficiency, calves at the beef industry are weaning at three to five months of age. The advantages of early weaning include induction of involution and increase in productivity, control of calf feeding, and reduced quantity and quality of food required for cow feeding. However, early weaning impose a great stress on the cows as well as on the calve, resulting in a general decrease in body condition, increase of udder pressure, milk leakage and agony, all leading to susceptibility to diseases and negative effects on the cow's next breeding cycle and to substantial calf weight losses. Thus, the current practice of controlled early weaning in lactating animals in modern farming hampers considerably the welfare state and reproductive performance of the animals.

### Casein Peptides

Casein (CN) is the predominant protein in human and non-human mammal's milk. It has been previously defined as composed of three fractions, α, β and γ, according to their electrophoretic mobility. Today, casein is defined according to the amino acid sequences of each of the subgroups αS1, αS2, β and κ (Engel et al. 1984. J. Dairy Sci. 67:1607-1608).

Enzymatic hydrolysis of casein liberates peptides that may contribute to the health and proper development of young and that serve as local regulators of mammary gland function (Silanikove et al. 2000 Life Sci. 67:2201-2212; Shamay et al. 2002 Life Sci. 70:2707-2719). U.S. Pat. No. 6,391,849 discloses casein-derived proteose-peptones that act as calcium chelators, and their use in controlling physiological changes in a mammary gland, including transient and persistent cessation of milk production, and prevention, treatment and reversal of infections. Proteose-peptones (PPs), also known as casein phosphopeptides (CPP), are a group of boiling-resistant peptides constituting about a third of whey proteins, which are principally the products of the activity of plasmin on β-casein and αs1- and αs2-casein (Andrews 1983 J. Dairy Res. 50:45-55).

Casein phosphopeptides have been shown to possess the unique property of being able to bind macroelements such as Ca, Mg, and Fe, along with trace elements such as Zn, Ba, Cr, Ni, Co and Se, which may be solubilized in the small intestine and therefore available for absorption. As such, CPPs are used as additives in beverages and infant food, and in dental medicaments (see, e.g., European patent No. EP 0090406; U.S. Pat. Nos. 5,130,123; 5,227,154).

It has previously been shown that a peptide derived from the activity of plasmin on β-casein (β-CN) down-regulates milk secretion in cows and goats (U.S. Pat. No. 6,391,849). The activity of this peptide was correlated with its ability to block potassium channels in the apical membranes of mammary epithelia (Silanikove et al., supra). It was also shown that injection of casein hydrolyzates (CNH) into the udder of a goat or a cow mimics the natural phenomenon of involution, inducing a local inflammatory response and loss of tight junction (TJ) integrity, followed by rapid drying-off mammary secretion (Shamay et al., supra; Shamay et al 2003 J. Dairy Sci. 86:1250-1258). The process induced by CNH was more rapid and synchronized than that induced at natural drying-off. These results indicate that it is possible to significantly reduce the time required for involution. The inventor of the present invention and co-workers have also shown that it is possible to shorten or omit the dry period without affecting the milk yield in the subsequent lactation (International Patent Application Publication No. WO2006/117784).

There remains an unmet need for methods and compositions to improve reproductive efficiency of a herd without negatively affecting the livestock welfare.

### SUMMARY OF THE INVENTION

The present invention relates to methods and compositions for improving the reproductive efficiency or productivity of a livestock herd and for inducing early weaning without negatively affecting the livestock welfare. Particularly, the present invention discloses methods and compositions for estrus induction and synchronization of ovulation in a herd, leading to the reduction of anestrus intervals, increase of conceiving rate and thus to an increase in reproduction rate. The present invention further provides methods for inducing early and stress-free weaning, leading to increased productivity of a herd.

The present invention is based in part on the unexpected discovery that applying casein-derived peptides (proteose-peptones) to a lactating livestock animal results in estrus induction in the animal.

Thus, according to one aspect, the present invention provides a method for estrus induction in a lactating livestock animal comprising administering to the animal an effective amount of at least one peptide derived from casein.

According to certain embodiments, the estrus occurs from about 2 days to about 120 days after peptide administration, preferably from about 2 days to about 50 days after the peptide administration. According to other embodiments, the method further comprises inseminating the lactating animal. According to one currently preferred embodiment, conception rate is at least 70%, preferably about 75%, more preferably about 80% or more.

According to certain embodiments, the method of estrus induction comprises administering the at least one casein-derived peptide of the invention from about 70 days before parturition to about 150 days after parturition. According to other embodiments, the peptide is administered from immediately after parturition to about 150 days after parturition, preferably immediately after parturition to 90 days after parturition, more preferably immediately after parturition to 60 days after parturition, most preferably immediately after parturition to 30 days after parturition.

The method of the present invention is effective in inducing estrus in any lactating animal. According to certain currently preferred embodiments, the method of the present invention is directed to estrus induction in livestock animals grown for meat or milk production including cows, goats, sheep, swine, camels and buffaloes.

Estrus induction according to the method of the present invention may be applied to an individual livestock animal as well as to a plurality of lactating animals in a herd, as to synchronize the estrus of all the treatment-receiving animals. Estrus synchronization in a herd is highly desirable, as it impart synchronization of subsequent steps in a herd life cycle, including insemination, calving, weaning, feeding program etc., and thus increasing the management efficiency.

Thus, according to certain embodiments, the at least one casein-derived peptide is applied to a plurality of lactating livestock animals in a herd, thereby synchronizing the estrus of the lactating animals in the herd. According to certain embodiments, the herd is of beef cattle. According to other embodiments, the herd is of dairy cattle. According to yet further embodiments, the herd is of swine.

According to other embodiments, estrus synchronization in the herd enables synchronized insemination According to certain embodiments, the method of estrus synchronization in a herd further comprises inseminating the animals. According to one embodiment, conception rate after insemination is at least 70%, preferably 75%, more preferably 80%. According to yet further embodiments, estrus synchronization in the herd results in synchronized parturition in the herd.

According to yet another aspect, the present invention provides a method for inducing a conception rate of at least 70% in a herd, comprising administering to a plurality of lactating animals in a herd an effective amount of at least one peptide derived from casein. According to certain embodiments, the conception rate is at least 75%, preferably 80% or more.

The peptide derived from casein can be obtained by hydrolysis of casein, or it can be a synthetic peptide. Any combination of casein-derived peptides, natural or synthetic, can be used according to the teaching of the present invention.

According to certain embodiments, the peptide is casein-derived phosphopeptide. According to one embodiment, the phosphopeptide derived from casein comprises an amino acid sequence as set forth in SEQ ID NO:1, and analogs, derivatives or fragments thereof. According to further embodiments, the phosphopeptide is selected from the group consisting of a phosphopeptide derived from β-casein, a phosphopeptide derived from αS1-casein and a phosphopeptide derived from αS2-casein. According to certain currently preferred embodiments, the phosphopeptide derived from β-casein comprises an amino acid sequence as set forth in SEQ ID NO:2 and analogs, derivatives or fragments thereof. According to additional currently preferred embodiments, the phosphopeptide derived from αS1-casein comprises an amino acid sequences as set forth in SEQ ID NO:3 and analogs, derivatives or fragments thereof. According to yet other currently preferred embodiments, the phosphopeptide derived from αS2-casein is selected from a peptide comprising an amino acid sequences as set forth in SEQ ID NO:4 and a peptide comprising an amino acid sequences as set forth in SEQ ID NO:5 and analogs, derivatives or fragments thereof.

Without wishing to be bound by any particular theory or mechanism of action, estrus synchronization by casein-derived peptides may be attributed to a change in the endogenous hormonal system towards secretion of estrus-inducing hormones.

The teachings of the present invention are advantageous over previously known hormonal methods for estrus synchronization, providing easy to apply methods that have no adverse effects. Moreover, the methods of the present invention contribute to the general health of the animal, improving the corporal condition as well as reducing stress factors, thus providing for the comfort and increased welfare of the animals.

The peptides of the invention can be administered by any suitable method as is known in the art. According to certain embodiments, the administration method is selected from the group consisting of systemic administration and direct administration into a mammary gland. According to certain currently preferred embodiment, the method comprises administration into a teat canal of the mammary gland of the lactating animal. The at least one peptide can be administered to one or more mammary glands, including simultaneous administration to all mammary glands of the animal. Administration to the teat can be performed by intracanal injection, by application of patch comprising the peptide of the invention, by soaking the teat in a composition comprising the at least one peptide and the like.

Single administration as well as multiple administrations is contemplated. The present invention now discloses that a single administration of at least one casein-derived peptide suffice to induce estrus synchronization. According to other embodiments, the peptide is administered between one to five times at intervals of from about 6 hours to about 24 hours.

According to another aspect, the present invention provides a method for inducing stress-free weaning of a suckling offspring comprising administering to a lactating animal at least one day prior to weaning an effective amount of at least one peptide derived from casein.

The weaning method of the present invention enables leaving the suckling offspring with his mother and thus significantly reducing the stress imposed by weaning on both the mother and the suckling offspring. It is a common experience for young calves, after being removed to a feedlot, to cry out for their mothers and to constantly walk around the feedlot in a semi-panicked and stressed state. During this stressful time period, calves often experience no gain in weight due to their unfamiliarity with the surroundings and their reluctance to eat conventional feed. The mothers suffer pain due to the abrupt cessation of suckling, pronounced by an increase in walking and reduction in lie-down periods. The stressful conditions and the reduction in the amount of food consumed by the animals, often results in such animals getting sick, and in a reduction in the overall body condition score. Relieving the stress by leaving the calves with their mother results in no weight loss of the calves and even in weight gain.

Weaning according to the teaching of the present invention can be induced at any stage of suckling, without negatively affecting the mother's welfare. Without wishing to be bound to any specific model or mechanism of action, this phenomenon may be attributed to the effect of casein-derived peptides on the hormonal balance of the lactating animal towards estrus, disclosed in the present invention for the first time.

According to certain embodiments, early weaning is commenced from about 2 weeks to about 15 weeks after parturition, preferably from about 4 weeks to about 8 weeks after parturition.

According to other embodiments, the at least one casein-derived peptide is administered between about 1 day to about 10 days prior to the scheduled weaning, preferably at about 3 days prior to weaning.

Administration of a single dose as well as of repeated doses of the at least one casein-derived peptide into a mammary gland can be applied. Typically, to obtain a stress free weaning, administration is repeated at least once, preferably between 1-10 times, more preferably 1 to 3 times, at an interval selected from the group consisting of about 6 hours, about 8 hours, about 12 hours, about 16 hours, about 20 hours and about 24 hours during 1 to 10 days, preferably 1 to 3 days, more preferably 1 day. According to certain currently preferred embodiments, the peptide is administered only once. After peptide administration the offspring may be separated from his mother for short periods of time, to facilitate the weaning process. Without wishing to be bound to a specific mechanism of action or theory, the weaning process is enhanced due to a change in the milk composition induced by the casein derived peptide, which result in a reduced suckling desire of the offspring. According to certain embodiments, the offspring is separated from his mother for a period of from about 5 h to about 3 days, preferably from about 5 h to about 2 days, more preferably from about 5 h to about 24 h.

According to yet another aspect, the methods for estrus synchronization and early weaning of the present invention may be used with a novel method for raising a livestock herd, and more particularly cattle, wherein there is an increase in the productivity of the herd resulting from an increase in reproductive rate, i.e. the number of calves per year, and in the quantity and/or quality of meat produced, without negatively affecting, or even improving, the livestock welfare. Livestock management according to the teaching of the present invention also may result in an overall reduction in the costs involved in the herd raising process by reduction in the practice of administering hormonal supplements to achieve estrus synchronization and by reducing the cost of special feed required after parturition to keep the cattle body condition balanced after parturition and breeding. That is, this aspect of the present invention combines the use of estrus synchronization and early weaning as disclosed herein within the context of a novel livestock management program. This novel livestock management also enables determining a seasonal calving program, as well as livestock-agriculture field planning, specifically in arid and semi-arid zones.

Other objects, features and advantages of the present invention will become clear from the following description.

### DETAILED DESCRIPTION OF THE INVENTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

### DEFINITIONS

As used herein, the term “casein” refers to the predominant protein in non-human mammals and human milk, comprising the subgroups αS1, αS2, β and κ.

As used herein, the term αS1, αS2 and β-casein refers to αS1, αS2 and β-casein protein of a mammal, including, but not limited to, livestock mammals (e.g., cow, sheep, goat, swine, mare, camel, deer and buffalo) human beings and marine mammals.

The term “peptide” is used throughout the specification to designate a linear series of amino acid residues connected one to the other by peptide bonds. The peptide according to the principles of the present invention is other than the intact protein.

As used herein, the term “phosphopeptide” designates a phosphorylated peptide in form of a conjugated peptide in which the non-peptide portion is a residue of phosphoric acid. In particular the expression “casein phosphopeptide” or “CPP” or “proteose-peptone” designates a phosphopeptide containing a casein fragment.

The peptides used in the methods of this invention preferably have an average molecular weight of from about 1,000 to about 10,000 Dalton, preferably from about 1,000 to about 5,000 Dalton. The invention particularly contemplates peptides having between 10-50 amino acid residues in total. The present invention also contemplates proteins in which the core motif sequence, e.g. the amino acid sequences set forth in SEQ ID NO:1, is artificially implanted within a sequence of a polypeptide, such as peptides manufactured by recombinant DNA technology or by chemical synthesis. The peptides can be obtained by hydrolysis of casein to yield a mixture of peptides. According to the teaching of the present invention, a mixture of the peptides can be used, or the mixture can be further purified by any protein purification method known in the art to obtain the isolated peptides. Alternatively or additionally, the peptides of the present invention can be cleaved by chemical agents such as, for example, CNBr or other cleaving agents known in the art, to yield a mixture of peptides, which can be further purified to obtain isolated peptides.

The peptides used in the methods of the present invention can also be synthesized using methods well known in the art including chemical synthesis and recombinant DNA technology. Synthesis can be performed in solution or by solid phase peptide synthesis as described by Merrifield (see J. Am. Chem. Soc., 85:2149, 1964).

In general, peptide synthesis methods comprise the sequential addition of one or more amino acids or suitably protected or derivatized amino acids to a growing peptide chain. Normally, either the amino or the carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth; traditionally this process is accompanied by wash steps as well. After all of the desired amino acids have been linked in the proper sequence, any remaining protecting groups (and any solid support) are removed sequentially or concurrently, to afford the final peptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide, and so forth.

As used herein the term “estrus” refers to the periodic state of sexual excitement in the female of most mammals, excluding humans, that immediately precedes ovulation and during which the female is most receptive to mating. The estrus cycles is regulated by hormonal interaction ruled by the hypothalamus-hypophysis-ovary-uterus axis.

The estrus cycle can be divided in three phases: 1) Follicular or luteal regression phase (proestrus); 2) periovulatory phase (estrus and metaestrus); and 3) luteal phase (diestrus). Day 0 of the estrus cycle is the estrus day, i.e. the day on which the estrus can be visibly seen. However, from the physiological point of view, the estrus cycle begins with the destruction of the corpus luteus and end with the destruction of the corpus luteus of the next cycle. The proestrus, which typically lasts for a 3-day period starts with the regression of the corpus luteus of the previous cycle and ends with the manifestation of the estrus. When the corpus luteus is destroyed, there is a fall in progesterone levels and, later on, a luteal tissue loss; in this process, the prostaglandin F2α (PGF2α) of a uterine origin is the main luteolytic agent in domestic animals and most rodents. As a result of the decline in progesterone levels, the negative feedback of this hormone at the hypothalamus level decreases as well, and the pulsatile frequency of the gonadotrophic hormones (FSH and LH) starts to increase, stimulating the follicular growth with the development of a large follicle and the increase in estradiol levels. When estrogens reach a certain level, the receptivity to the male becomes stimulated and the estrus cycle starts.

The estrus and metaestrus phase starts with the receptivity to the males and involves all changes allowing for the ovulation and the beginning of the corpus luteus formation. During the estrus, lasting from about 16 to about 24 h, a cow would typically show restlessness and anxiety, bellows frequently and loses appetite. In the case of dairy cows, milk production becomes affected. The cows show vaginal mucus discharge, that its smell appeals and excites the bull (presence of pheromones), vulva edema and an increase of the myometrial tone of the uterus, easily detected by transrectal palpation.

During this phase, the high concentrations of estrogens reach the stimulation threshold of the hypothalamic cyclic center, stimulating the hypothalamic neurons to produce the gonadotropin releasing hormone (GnRH) peak and consequently, the LH peak. As regards the FSH, its secretion decreases as a result of the negative feedback of the estrogens and the inhibin, except for the moment when the LH preovulatory peak occurs where a FSH peak can appear. Later, 4 to 12 hours after the LH wave, basal concentration and the FSH pulse width increase, and this process is related to the first wave of follicular growth.

From 12 to 24 hours after estrus beginning, the cow's nervous system becomes refractory to estradiol and the psychic manifestations of the estrus come to halt.

The period immediately following the end of the estrus is called metaestrus (6 days). During this period, the ovulation of the cow occurs, unlike other species that ovulate during the estrus, giving rise to cell organization and the development of the corpus luteus. Ovulation occurs 28 to 32 hours after beginning of the estrus and is unleashed by the LH preovulatory peak. Ovulation is followed by a deep bleeding and the follicle is filled with blood and becomes a hemorrhagic body.

While the corpus luteus is formed (luteinization), a series of morphological and biochemical changes occur, allowing follicular cells to transform into luteal cells. These changes end on the seventh day with the formation of a functional corpus luteus.

The diestrus phase is characterized by the predominance of the corpus luteus. The maintenance of the corpus luteus as well as the progesterone synthesis are related to the progesterotrophic and luteotrophic LH hormone.

Other hormones taking part in the progesterone synthesis are FSH and prostaglandin 2 (PG2). The FSH hormone would apparently join to receptors located in the corpus luteus and would cause an increase in progesterone secretion. As regards PG2, in addition to stimulating luteal cells to produce progesterone, it may increase the blood flow at the ovarian level, having a positive effect on the synthesis and secretion of progesterone.

If the ovum is not fertilized, the corpus luteus remains functional until day 15-20, after which regression starts in order to prepare for a new estrus cycle.

As used herein the term “anestrus” refers to an interval of sexual activity between two periods of estrus in female mammals that breed cyclically.

The terms “postpartum interval (PPI)”, as used herein refer to the time between parturition to the beginning of estrus (the proestrus).

The terms “calving to first postpartum ovulation”, and “parturition-conception interval” are used herein interchangeably, and refer to the time between parturition to the late phase of the estrus (metaestrus).

The term “puerperium” as used herein refers to the period right after parturition, i.e. the approximate six-week period lasting from parturition to the return of normal uterine size.

The term “suckling” as used herein refers to the feeding behavior of very young mammals, and is the drawing of milk into the mouth of the young mammal from the nipple of a mammary gland. According to the current used practices, suckling can be ad libitum, or restricted to specific times during the day or restricted during the all day.

As used herein the terms “dry period” or “period of dry cow” refer to the phase before parturition in which milking is ceased. According to currently used practices, applying a dry period is necessary to complete the process of involution, after which the milk secretion capacity is restored toward parturition.

The terms “weaning” and “calf removal” are used herein interchangeably, referring the separation of a suckling offspring from its mother, while the mother is lactating.

As used herein, the term “early weaning” refers to weaning before a natural process of milk cessation and involution occurs. Specifically, the term “early weaning” refers to weaning from about 2 weeks to about 15 weeks after parturition. Weaning from about 2 weeks to less than about 8 weeks after parturition is also referred to as “super early weaning”. As used herein, the term “livestock-agriculture field planning” refers to agricultural land management comprising altering the use of the land between growing agricultural crops and using the land as a pasture.

As used herein, the term “body condition score” refers to a number that represents the body reserves of a livestock animal, particularly a cow, at a certain time point. A body condition score is assigned by visual observation of the cow's rump area—primarily the region delimited by the hip bones (tuber coxae), the pinbones (tuber ischii) and the tail-head. The amount of “covering” over the vertebrate of the back is also used in giving a score. Cows are usually ranked on a scale from 1 to 5. Extremely thin cows are assigned a score of 1 and extremely fat cows are assigned a score of 5.

As used herein, the term “livestock” refers to animals, such as beef cattle, dairy cattle, sheep, goats, horses, swine, buffaloes and camels raised for food or for the production of food for home use or for large scale production and or for profit, particularly on a farm.

As used herein, the terms “livestock welfare” or “welfare in animal farm” or “livestock comfort” refer to the prevention of suffering and increasing the presence of positive feelings, usually called comfort or pleasure, resulting from, inter alia, an increase in resting time, in periods of lie down, and in ruminating time, and a decrease in metabolic need, udder pressure, teat leakage, mastitis incidence and other diseases and lameness effect due to high milk yield.

The methods of the present invention may be applied to any mammalian livestock animal of which the female is going through estrus cycle. As an example, and without intending to be limiting, the present invention refers to cattle herds. According to certain currently preferred embodiments, the present invention refers to beef cattle herds.

Management of a cattle herd can be aimed at meat production and/or milk production. The productive cycle of a breeding cow can be divided into four periods: period of dry cow; parturition preparatory period; parturition; and lactation. Each of these periods has specific nutritional requirements and hormonal characteristics. The pregnancy of the animals involves a substantial cost, since the requirements of the last month of gestation are higher than those applicable to a non-pregnant animal. Synchronization of parturition time within a herd is highly beneficial. If calving is taken as Day 0 of the calendar year, the more synchronic the 0 Days of a herd, the easier it would be to apply a beneficial management regime. Fodder supply can be adjusted to the nutritional needs of the herd, and as a result, the physiological state of the cattle would be improved and at the same time economic issues would be addressed. Synchronization of parturition is directly depended on estrus synchronization.

Moreover, attempts to control PPI, estrus and the sequential progression of follicles and ovulation responses by manipulating nutrition and by hormone treatment following calving in dairy cattle have proved equivocal. The accelerated involution in dairy cattle following administration of casein derived peptides, which leads to a lesser udder engorgement and risk of intramammary infection during and immediately after the dry-period results in a better animal comfort and body conditions during calving and postpartum. Without been bound to a specific mode of action it has been surprisingly discovered that administration of casein derived peptides control PPI in dairy cattle, mostly by reducing PPI which is followed by a rapid commencement of ovulation and expression of estrus.

Thus, according to one aspect, the present invention provides a method for estrus induction in a lactating animal comprising administering to the animal an effective amount of at least one peptide derived from casein.

According to certain embodiments, the at least one casein-derived peptide is applied to a plurality of lactating livestock animals in a herd, thereby synchronizing the estrus of the plurality of lactating animals.

As used herein the phrase “peptides derived from casein” or “casein derived peptide” refers to peptides which are cleavage products of casein (referred to herein as peptides derived from natural casein), synthetic peptides, chemically synthesized to correspond to amino acid sequences of the casein units (referred to herein as synthetic peptides derived from casein), and peptides similar (homologous) to casein, for example, peptides characterized by one or more amino acid substitutions, insertions or deletions, such as, but not limited to, permissible substitutions, provided that at least 70%, preferably at least 80%, more preferably at least 90% similarity is maintained, and functional homologues thereof. The terms “homologues” and “functional homologues” as used herein mean peptides with any insertions, deletions and substitutions which do not affect the biological activity of the peptide as described herein.

Any combination of casein-derived peptides can be used according to the teaching of the present invention. The phrase “combination” is defined as any of the above-mentioned casein derived peptides, natural or synthetic, combined in a mixture with one or more additional, non-identical peptides. As used herein, the term “mixture” is defined as a non-covalent combination of peptides existing in variable proportions to one another.

According to certain embodiments, the peptide derived from casein is a phosphopeptide comprising the active motif Ser(p)-Ser(p)-Ser(p)-Glu Glu (SEQ ID NO:1), and analogs, derivatives or fragments thereof. It should be understood that any peptide comprising this motif—whether derived from casein, from a protein other than casein, synthetically synthesized or produced by recombinant technology—which retains the biological activities of the peptides as are described herein, is also encompassed within the scope of the present invention. The phosphopeptides of the present invention are exemplified by peptides having an amino acid sequence as set forth in any one of SEQ ID Nos. 2-5, as listed below:

| SEQ ID | | Derived | Residue |
|---|---|---|---|
| NO. | Sequence | from | number |
| SEQ ID | RELEELNVPGEIVES(P)LS(P)S(P)S(P)EESITR | β-casein | 1-25 |
| NO: 2 | | | |
| SEQ ID | QMEAESIS(P)S(P)S(P)EEIVPDSVEQK | αS1-casein | 59-79 |
| NO: 3 | | | |
| SEQ ID | KNTMEHVS(P)S(P)S(P)EESIISNETYK | αS2-casein | 1-21 |
| NO: 4 | | | |
| SEQ ID | NANEEEYSIGS(P)S(P)S(P)EESAEVATEEVK | αS2-casein | 46-70 |
| NO: 5 | | | |

The term “analog” includes any peptide comprising altered sequence by amino acid substitutions, additions, deletions, or chemical modifications of the peptides of the invention and which retain the biological activity of the peptide. By “amino acid substitutions”, it is meant that functionally equivalent amino acid residues are substituted for residues within the sequence resulting in a silent change. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such substitutions are known as conservative substitutions. Additionally, a non-conservative substitution can be made in an amino acid that does not contribute to the biological activity of the peptide. It will be appreciated that the present invention encompasses peptide analogs, wherein at least one amino acid is substituted by another amino acid to produce an active analog of a peptide of the invention having increased stability or longer half-life as compared to the peptide listed herein.

While the amino acid residues of the peptide sequences set forth in SEQ ID NO:1 to 5 are all in the “L” isomeric form, residues in the “D” isomeric form can substitute any L-amino acid residue so long as the peptide analog retains its activity. Methods of producing a retro-inverso D-amino acid peptide analog where the peptide is made with the same amino acids as disclosed, but at least one or more amino acids, including all amino acids are D-amino acids, are well known in the art. When all of the amino acids in the peptide analog are D-amino acids, and the N- and C-terminals of the peptide analog are reversed, the result is an analog having the same structural groups being at the same positions as in the L-amino acid form of the peptide. However, the peptide analog is more stable to proteolytic degradation and is therefore useful in many of the applications recited herein.

The term “derivative” refers to a peptide having an amino acid sequence that comprises the amino acid sequence of the peptide of the invention, in which one or more of the amino acid residues is subjected to chemical derivatizations by a reaction of side chains or functional groups, where such derivatizations do not destroy the activity of the peptide derivative. Chemical derivatization of amino acid residues include, but are not limited to, glycosylation, oxidation, reduction, myristylation, sulfation, acylation, acetylation, ADP-ribosylation, amidation, cyclization, disulfide bond formation, hydroxylation, iodination, and methylation.

The peptide derivatives according to the principles of the present invention also include bond modifications, including, but not limited to, CH₂—NH, CH₂—S, CH₂—S═O, O═C—NH, CH₂—O, CH₂—CH₂, S═C—NH, CH═CH, and CF═CH and backbone modifications. Peptide bonds (—CO—NH—) within the peptide may be substituted, for example, by N-methylated bonds (—N(CH₃)—CO—); ester bonds (—C(R)H—C—O—O—C(R)—N); ketomethylene bonds (—CO—CH₂—); α-aza bonds (—NH—N(R)—CO—), wherein R is any alkyl group, e.g., methyl; carba bonds (—CH₂—NH—); hydroxyethylene bonds (—CH(OH)—CH₂—); thioamide bonds (—C═S—NH—); olefinic double bonds (—CH═CH—); and peptide derivatives (—N(R)—CH₂—CO—), wherein R is the “normal” side chain, naturally presented on the carbon atom. These modifications can occur at any of the bonds along the peptide chain and even at several (2-3) at the same time.

The present invention also encompasses those peptides in which free amino groups have been derivatized to form amine salts, including but not limited to hydrochlorides, p-toluene sulfonyl groups, carbobenzoxy groups, t-butyloxycarbonyl groups, chloroacetyl groups or formyl groups. Free carboxyl groups may be derivatized to form, for example, salts, methyl and ethyl esters or other types of esters or hydrazides. Free hydroxyl groups can be derivatized to form, for example, o-acyl or o-alkyl derivatives. The imidazole nitrogen of histidine can be derivatized to form N-im-benzylhistidine.

Also included as chemical derivatives are those peptides, which contain one or more naturally occurring amino acid derivatives of the twenty standard amino acid residues. For example: 4-hydroxyproline can be substituted for proline; 5-hydroxylysine can be substituted for lysine; 3-methylhistidine can be substituted for histidine; homoserine can be substituted for serine; and ornithine can be substituted for lysine. The peptides can also contain non-natural amino acids. Non-limiting examples of non-natural amino acids are norleucine, ornithine, citrulline, diaminobutyric acid, homoserine, homocysteine, isopropyl Lys, 3-(2′-naphtyl)-Ala, nicotinyl Lys, amino isobutyric acid, and 3-(3′-pyridyl-Ala). The peptides may also contain non-protein side chains. In addition to the above, the peptides of the present invention can also include one or more non-amino acid monomers or oligomers (e.g., fatty acids, complex carbohydrates, and the like). Also encompassed is any peptide having one or more additions of amino acid residues relative to the sequences of the peptides listed hereinabove, so long as the requisite activity and preferably the molecular weight are maintained. The amino acid residues can be added at the amino terminus and/or carboxy terminus and/or along the peptide sequence.

A peptide derivative according to the present invention can also be a cyclic peptide. Cyclization can be obtained, for example, through amide bond formation, e.g., by incorporating Glu, Asp, Lys, Orn, di-amino butyric (Dab) acid, di-aminopropionic (Dap) acid at various positions in the chain (—CO—NH or —NH—CO bonds). Backbone to backbone cyclization can also be obtained through incorporation of modified amino acids of the formulas H—N((CH₂)ₙ—COOH)—C(R)H—COOH or H—N((CH₂)ₙ—COOH)—C(R)H—NH₂, wherein n=1-4, and further wherein R is any natural or non-natural side chain of an amino acid. Backbone to side-chain and side-chain to side-chain cyclizations are also contemplated.

Cyclization via formation of S—S bonds through incorporation of two Cys residues is also possible. Additional side-chain to side chain cyclization can be obtained via formation of an interaction bond of the formula —(—CH₂—)ₙ—S—CH₂—C—, wherein n=1 or 2, which is possible, for example, through incorporation of Cys or homoCys and reaction of its free SH group with, e.g., bromoacetylated Lys, Orn, Dab or Dap.

The term “fragment” as used herein refers to a peptide having one or more deletions of amino acid residues relative to the sequences of the peptides listed herein, so long as the requisite activity is maintained. The amino acid residues may be deleted from the amino terminus and/or carboxy terminus and/or along the peptide sequence.

Peptide fragments can be produced by chemical synthesis, recombinant DNA technology, or by subjecting the peptides listed herein to at least one cleaving agent. A cleaving agent can be a chemical cleaving agent, e.g., cyanogen bromide, or an enzyme, e.g., an exoproteinase or endoproteinase. Endoproteinases that can be used to cleave the peptides of the invention include trypsin, chymotrypsin, papain, V8 protease or any other enzyme known in the art to produce proteolytic fragments.

As described hereinabove, the peptides of the present invention can be obtained by hydrolysis of casein, or the peptides can be obtained synthetically.

Hydrolysis of casein can be performed by any method as is known to a person skilled in the art. Enzymes used in hydrolysis of casein include enzymes derived from animals, for example, pancreatin, trypsin, chymotrypsin, neutrase, alcalase, pepsin, carboxypeptidase, cathepsins, and the like; enzymes derived from plants, for example, papain, bromelain, and the like; and enzymes derived from microorganisms, for example, lactobacillus, yeast, fungi, Bacillus subtilis, Actinomyces, Aspergilus, Micrococcus caseolyticus and the like. Typically, hydrolysis of casein is performed by digestion with trypsin. Non-digested casein is then separated from the peptide-containing solution, which is further purified from other impurities by a suitable method as is known in the art.

Planned reproductive management program has many advantages and benefits, as is known to those skilled in the art, and can be generally described to include the following: it allows for planning of calving timing and dates—cow that are inseminated at early age that is early in the season have higher lifetime calf production than those that calve late; enables livestock-agriculture field planning for appropriate use of the field and improves the rotational grazing, ensuring an efficient distribution of fodder to meet the physiological feeding needs of the herd individuals, wherein cows reaching calving with good body conditions (body condition score of 2.75-3.50) exhibit better estrus induction and become pregnant in a finite breeding season; enables the design of calving and inseminations which optimizes the work of the personnel and the conception rate; decreases the number of bulls per herd, allowing for the investment in bulls with superior genetics and quality; improves the work with the calves, allowing for their distribution in homogenous groups; enhances the sustainability of the estrus system, thus avoiding dependence on natural periods; facilitates compliance with the vaccination program and improves its efficiency.

Applying the method of estrus induction according to the present invention allow for the estrus to occur immediately at the end of the puerperium period, and thus shortening the postpartum interval (PPI). Shortening the PPI is highly desired. It has been shown that the pregnancy percentage falls linearly when the parturition-estrus interval increases from 60 to 120 days. In addition, this parturition-first estrus relationship shows that calf kilograms decrease considerably when such interval is extended. As a result of estrus induction and synchronization, more cows calve early the next year and in subsequent years of synchronization in accordance with management efficiency.

Naturally, management decisions and procedures have some influence on the parturition-conception interval but the latter is mainly determined by the following factors: the reestablishment of ovarian cycles after calving; the occurrence of the estrus at the proper time of the cycle; the pregnancy rate after the service. The pregnancy rate increases almost linearly when the estrus fertility increases. The slope depends on the parturition-estrus interval, and it increases when this interval shortens.

Without wishing to be bound to any theory or mode of action, the estrus induction obtained by administering at least one peptide derived from casein according to the methods of the present invention may be attributed to a change in the type and timing of hormone secretion. During the post-calving period, lactating females suffer an important change in their energy balance prior to the onset of the normal ovarian cycles. This negative energy balance is largely caused by the loss of energy resulting from lactation, larger than the energy that can be regained with food. This negative balance is associated with the hormonal plasma profiles determining a lower activity in the follicular dynamics and resulting in lack of estrus and ovulation. Cows with a body condition score below than 2.5 have reduced LH pulse frequency, reduced ovarian activity and delay return to estrus post parturition. The reestablishment of LH pulsatile secretion after calving produces the restart of the normal follicular dynamics. The early beginning of the estrus cycles becomes a determining factor of an early conception. The moment of the first ovulation determines and limits the number of estrus cycles that are likely to occur before the first insemination, and the higher the number of estrus before the 60-day post-calving period, the higher the chance of conception at the first insemination (for example, 2.60 and 1.75 insemination attempts per conception for cows of 0 and 4 estrus respectively before the 60-day post-calving period). The objective of the producers should be to fertilize the cow in the first or second insemination; otherwise, the number of open cow days would increase and the calving-conception period would be longer, leading to a decrease in fertility rate with the resulting production losses. Casein-derived peptides provide a means to induce the desired estrus and control the postpartum interval and to improve the negative energy balance described above.

Productivity of a herd depends not only on the number of offspring but also on the weight gained until slaughtering. One parameter determining a calve weight is the postpartum interval (PPI) as described hereinabove; another significant parameter is the stress imposed on the calve during weaning. Naturally, weaning occur in a cattle at about six to seven months from calving. The modern management practice of a beef herd employs weaning at four to eight weeks. Early weaning has few advantages, including the following:
It aids in controlling the reproductive cycle.
Calves can grow to their genetic potential regardless of the mother's milk production.
It may be the key to more efficient feed utilization during times of drought or other periods of feed shortage.
It reduces fodder cost as 15 to 20% less energy is needed to feed early-weaned calves and their mothers as compared to cows nursing their calves, and early-weaned calves have better feed conversion.
It fits in with fall calving where heavy winter-feeding would otherwise be required.
Early weaning permits more cows to be carried on a limited feed supply.

However, early weaning imposes a high stress on the calves and on their mothers, resulting in reduced productivity and profit. In order to avoid substantial weight losses, high quality and expensive calf nutrition should be used; the stress decreases the mother's body condition score and negatively affects the next breeding cycle. Moreover, the anticipated gain in heavy milking due to the early weaning may be lost due reduced milk production by the stressed mother, and by a general decrease in the mother and offspring body condition.

The present invention now discloses a method for inducing stress free, early weaning of a suckling offspring comprising administering to a lactating animal effective amount of at least one peptide derived from casein.

The advantages of the method of the present invention is in that it enables determining the desired time for weaning, from few days to several months after calving and in that the offspring have the option to stay by their mothers, such that the weaning process is stress free for both the offspring and mothers, and no decrease in body weight and general conditions is observed.

In addition, the present invention now discloses that administering casein-derived peptides to a mammary gland of a lactating animal, particularly a cow, reduces the udder pressure, which is another factor that is known to cause a high stress, lack of comfort, susceptibility to diseases and reduced welfare to the mother.

The process of abrupt cessation of milking and posterior mammary involution causes agony to the lactating animal and leads to a change in their behavior. The last is shown by increase stepping and decrease in lying-down time. The rapid involution after the treatment with casein-derivate peptide, which decreases the milk synthesis in about 6 hours to 4 days, prevents this discomfort. This is shown by an increase in lying behavior and rumiantive time. Without wishing to be bound to any specific model or mechanism of action, this phenomenon may be attributed to the effect of casein-derived peptides on the udder pressure, which is largely reduced after application of the peptides of the invention.

Thus, the methods of the present invention provide an increased profitability, due to an increase in progeny weight, and by avoiding overfeeding.

Casein-derived peptides can be administered according to the methods of the present invention in any suitable pharmaceutical composition and formulation as is known to a person skilled in the art.

As used herein a “pharmaceutical composition” refers to a preparation of a casein-derived peptide as described herein, with other chemical components such as physiologically suitable carriers and excipients. The purpose of a pharmaceutical composition is to facilitate administration of a compound to an organism. The term “active ingredient” as used herein refers to a compound accountable for the biological effect. The terms “physiologically acceptable carrier” and “pharmaceutically acceptable carrier” which may be interchangeably used refer to a carrier or a diluent that does not cause significant irritation to an organism and does not abrogate the biological activity and properties of the administered peptide. The term “excipient” refers to an inert substance added to a pharmaceutical composition to further facilitate administration of a compound. Examples, without limitation, of excipients include various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Pharmaceutical compositions for use in accordance with the methods of the present invention may be formulated in conventional manner using one or more physiologically acceptable carriers comprising excipients and auxiliaries, which facilitate processing of the active ingredient into preparations which, can be used pharmaceutically. Proper formulation is dependent upon the route of administration chosen.

For injection, the peptides of the invention may be formulated in aqueous solutions, preferably in physiologically compatible buffers such as Hank's solution, Ringer's solution, or physiological saline buffer.

The preparations of peptides to be used with the methods of the present invention may be formulated for parenteral (intramammary) administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions suitable for use with the methods of the present invention include compositions wherein the active ingredients are contained in an amount effective to achieve the intended purpose. More specifically, an effective amount means an amount effective to induce estrus synchronization and early weaning.

### EXAMPLES

### Example 1

### Estrus Induction and Synchronization in Beef Cattle

### Purpose

The primary purpose of the present study is to induce and synchronize the return to estrus in beef cows after calving, by intracanal administration of casein derived peptides into each teat quarter. The secondary purpose is to reach successful fertilization in no more than 60 days after peptide administration, and to efficiently reach high body and performance score in the cows.

Successful outcome of the treatment is considered as the induction of the estrus stage up to 20 days following peptide administration of at least 80% of cows, and conception of at least 70% of the treated cows up to the third pulse.

### Study Design

The clinical trial is designed to be a two regimes (arms) case-control study. “Arm 30” refers to calves weaned 30 days postpartum date. “Arm 60” refers to weaning the calves 60 days postpartum.

“Cases” and “Controls” are beef cows enrolled at least 14 days before the expected calving date. Weaning is scheduled for both groups according to the planned Arm (30 or 60 days postpartum). Cases receive intramammary administration of casein-derived peptides, while controls do not to receive any intramammary administration. Both case and control cows live in the same herd, are of the same age (±180 days) being after calving (same calving date ±60 days) and are milking for the same period (±90 days) at least 14 days in parallel. For each regime, total of 30 cows are enrolled, 15 cases and 15 controls.

The treatment of casein derived peptides is administered 1-2 times a day during one day.

The inclusion criteria for cow participation in this study are: cows at late gestational stage, 2 weeks prior to the expected calving day; cows that have four functional quarters; pregnant cows—first or more pregnancies; cows with or without confirmed intramammary infection who have either or not received prior therapy; cows with no significant external teat lesion; no systemic therapy within the past 8 weeks; no feeding with antibiotic additives within the past 8 weeks; absence of co-morbidities considered to potentially influence the outcome of treatment in the judgment of the investigator.

The exclusion criteria for cow participation in this study are: prior immunotherapy within 8 weeks of study entry; prior antibiotic, hormone, anti-inflammatory and anabolic therapies, either systemic or by feeding within 8 weeks before study entry; concurrent alternative therapies; cows with active tuberculosis or other infectious disease in the judgment of the investigator; concurrent use of anabolic steroids, either systemic or by feeding; concurrent use of hormones, either systemic or by feeding.

The total duration of the clinical trial is around 120 days (17 weeks), including twenty two (22) visits to the site (herd), as follows:

First Period: baseline/screening visit procedures involve at least 6 visits from 15 days before calving up to 30 days following calving for all cows participating in the study. Visit to the study place is at 15 and 7 days before calving, at calving day and 3 visits before treatment.

Second Period: sampling, start of Arm 30, insemination and follow-up of Arm 30 involve at least 7 visits—from day 30 after calving up to day 52: one visit at day 30 for sampling and weaning, 3 consecutive days visits after weaning (days 31, 33, 34), and 3 visits (days 38, 45—insemination day, and 52) up to the start of Arm 60.

Third Period: start of Arm 60 and follow-up of Arm 30 involve at least 4 visits—from day 60 after calving up to day 64 (one visit at day 60 for sampling and weaning of Arm 60, 3 consecutive days visits after weaning (days 61, 63, 64)).

Fourth Period: end of follow-up of Arm 30 and insemination of Arm 60 involve at least 2 visits (days 68 and 75—insemination day). On day 75 from the calving day, the follow up for cases and controls of Arm 30 ends. Cows enrolled in Arm 60 are inseminated.

Fifth Period: end of follow-up of Arm 60 and end of the clinical trial involve 3 visits (days 82, 91 and 105).

During the study clinical examinations are performed in cases and controls, including udder examination, specific clinical systems, weight and pregnancy status. Milk is sampled to test fat percentage, protein percentage, lactose percentage, somatic cell counts, and for bacteriological examination. Blood is sampled to test mainly estrogen, progesterone, LH, FSH and prostaglandin.

Safety measures include vital signs, physical examination, incident diseases, assessments of adverse event and bacteriological tests.

At the end of the study, the following end points are measured: mammary secretion; conception rate by pulse; fertility rate; fertility by time after calving and/or weaning; cow weight after weaning; presence of infectious diseases.

Efficacy of cessation of milk secretion following administration of casein-derived peptides is measured as follows: high success: cessation within 1-4 days; success 5-8 days; low success 9-15 days; unsuccessful 16 or more days for cessation of milk secretion.

The PPI length is measured from the administration day of casein-derived peptides up to ovulation days. High success is considered as PPI less than 12 to 20 days; success 21 to 24 days; low success 25 to 29 days; unsuccessful 30 or more days of PPI length.

A successful conception rate is measured as follows: high success 80% or more; success 70% to 79%; low success 50% to 69%; unsuccessful 49% or less.

The clinical trial is stopped at any of the following conditions: completion of study; screening failure; refusal by herd's owner; Cow suffering intolerable adverse events; Calf suffering intolerable adverse events; violation of inclusion/exclusion criteria; any factor or condition that would in the opinion of the investigator necessitate withdrawal from the study.

### Example 2

### Calve Weaning

Abrupt cessation of suckling is associated with increased udder pressure, which results in milk leakage and agony to the milking animal. Thus, calf weaning is associated with stress for the mothers and the offspring, such stress leading to weight loss and susceptibility to diseases.

### Purpose

The primary purpose of this study of early weaning without separating the calf from the cow is to determine the extent to which a continues non-suckling post-weaning contact between beef cows and their calves reduces the negative effects of termination of milk feeding on cow and calf behavior and growth.

The secondary purposes are to measure behavioral distresses of cows and calves after weaning by step-walking and lying-down time and by changes in their weights, up to 15 days post-weaning.

### Study Design

The trial is designed to be a two regimes (arm) case-control study. “Arm 30” refers to calves weaning 30 days postpartum. “Arm 60” refers to calves weaning 60 days postpartum.

“Cases” and “Controls” are beef pregnant cows enrolled at least 14 days before the expected calving date, and managed to early weaning (either at 30 or 60 days postpartum). Cases are sampled to receive intramammary administration of casein-derived peptides. Controls do not receive any intramammary administration.

Inclusion and exclusion criteria for cows as well as study conditions are as described in Example 1 hereinabove. The treatment of casein-derived peptides is administered 1-2 times a day during one day.

The inclusion criteria for calf participation in this study are: calf born at a late gestational stage—at least at 230 days; calf able to walk immediately; calf with no significant external lesion; calf weight at least 50 Kg; calf being treated with antiparasites medications and antirespiratory vaccines; absence of co-morbidities considered to potentially influence the outcome of treatment in the judgment of the investigator.

The exclusion criteria for calf participation in this study are: castration during the study; calf concurrent alternative therapies; calf with active tuberculosis or other infectious disease in the judgment of the investigator.

The total duration of the clinical trial is around 120 days (17 weeks), including twenty (20) visits to the site (herd), as follows:

Baseline/screening visit procedures involve at least 4 visits from calving up to 30 days after calving for all participants in the study. One visit to the study place is made at calving day and 3 visits are made before treatment. Follow up visits are as described in Example 1 hereinabove up to the end of the fifth period.

At 15 days postpartum, cows are randomly allotted within parity and age to one of the 2 weaning dates, at 30 or 60 days after calving, and to receive (cases) or not (controls) an intramammary administration of casein-derived peptides. Cows and calves are weighed, and body condition score are assessed immediately after calving and before weaning. Body condition score is assessed on a scale of 0-5, on which 0 is severe emaciation and 5 is obese.

At weaning day (30 and 60) postpartum, cow-calf pairs are moved to a large dry-lot location. Cases cow-calf pairs are allocated at dry-lot no more than 8 pairs per dry-lot. Control cow-calf pairs are moved to a separate distant dry-lot.

Cows are fed with an energy diet by providing 90-100 MJ metabolizable energy per cow per day (this is close to 100% of the recommendation for a 460-kg beef cow producing about 7-8 Kg of milk and with no change in live weight). The diets comprise 60% grass silage and 40% concentrates (barley, soy bean, mineral, and vitamins; 16% crude protein). Before weaning and treatment date, cows are housed and fed in groups of not more than 8 cows according to calving date. Following the first day post-weaning, calves are fed with protein, vitamins and minerals to meet the standard requirements, and fed at nearly constant quantities within stage of growth. During the first post-weaning day calves receive water only.

Behavior in cases and controls is measured by a leg-mounted telemetry system. This system enables monitoring and recording of accumulative records of walling steps, lying times and lying periods. The sensor has data storage and transmission capabilities, and includes an integrated power source sufficient for at least 1 month of operation. Collected data are downloaded from the sensor once each week during the follow-up period.

Safety measures include vital signs, physical examination, incident diseases, and assessments of adverse events.

At the end of the study, the following end points are measured:

Efficacy of cessation of milk secretion following administration of casein-derived peptides is measured as follows: cessation within 1-4 days; success 5-8 days; low success 9-15 days; unsuccessful 16 or more days for cessation of milk secretion.

The evaluation of body condition score in cows from weaning to 30 days post-weaning is made as follows: high success—no change or increase in weight; lower score of 0 to 0.5 points—success; lower score of 0.5 to 1 point, low success; lower score of 1.0 or more points, unsuccessful.

The weight condition in cows from weaning to 30 days post-weaning is evaluated as follows: high success no change or increase; lower weight up to 5%, success; lower weight between 5% to 10%, low success; lower weight of 10% or more, unsuccessful.

The weight condition in calves from weaning to 30 days post-weaning is evaluated as follows: high success—increase of 10 or more percentage; increase of 5 to 9 percentage, success; no change up to 4 percentage increase, low success; and lower weight, unsuccessful.

The behavior in cows and calves is evaluated by walking steps from standard values as follows: high success—decrease in 10 or more percentage; success—decrease in 5 to 9 percentage; low success—no change or increase/decrease up to 4 percentage; unsuccessful—increase in 5 or more percentage.

The behavior in cows and calves is evaluated by lying time from standard values as follows: high success—increase in 10% or more; success increase in 5% to 9%; low success—no change or increase/decrease up to 4%; unsuccessful—decrease in 5% or more.

The clinical trial is stopped at any of the following conditions:

Completion of study; screening failure; refusal by herd's owner; Cow suffering intolerable adverse event; Calf suffering intolerable adverse event; violation of inclusion/exclusion criteria; any factor or condition that would in the opinion of the investigator necessitate withdrawal from the study.

### Example 3

### Estrus Induction and Synchronization in Dairy Cows

### Purpose

The primary purpose of the present study is to induce and synchronize the return to estrus in dairy cows after calving, by intracanal administration of casein-derived peptides into each teat quarter prior to the dry-off period. The secondary purpose is to reach successful fertilization in no more than 105 days after the administration date.

Successful outcome of the treatment is considered as the induction of the estrus stage up to 20 days following administration of at least 80% of cows, and conception of at least 70% of the treated cows up to the third pulse.

### Study Design

The clinical trial is designed to be a case-control study.

“Cases” and “Controls” are dairy cows enrolled at least 14 days before the expected dry-off date. Cases are sampled to receive intramammary administration of casein-derived peptides or casein hydrolyzate (CNH), and controls are sampled not to receive any intramammary administration.

Both case and control cows live in the same herd, are of the same age (±180 days) being after calving (same calving date ±60 days) and are milking during the same period (±90 days) for at least 14 days in parallel.

The treatment of casein-derived peptides (or CNH) is administered 1-2 times a day during one day. The inclusion and exclusion criteria for dairy cow participation in this study are those defined in Example 1 hereinabove.

The total duration of the clinical trial is around 120 days (17 weeks), including thirteen (13) visits to the site (herd) as follows:

First Period: baseline/screening visit procedures involve at least 2 visits from 15 days before drying off to treatment day for all participants in the study. Visits to the study place takes place at 15 and 7 days before treatment at the onset of the dry-off period.

Second Period: sampling and treatment day includes blind enrolment of cases and controls and intramammary treatment.

Third Period: during the dry-off period, at least 4 visits are taken from day 1 at the treatment day to day 60 or calving day.

Fourth Period: calving and insemination involve at least 6 visits up to the end of follow-up. During the fourth period one visit is at the calving day, and at least three visits during the following three weeks for the inseminations and follow up.

During the study clinical examinations is performed in cases and controls including udder examination, specific clinical systems, weight and pregnancy status. Milk is sampled to test fat percentage, protein percentage, lactose percentage, somatic cell counts, and bacteriological examination. Blood is sampled to test mainly estrogen, progesterone, LH, FSH and prostaglandin.

Safety measures include vital signs, physical examination, incident diseases, assessments of adverse event and bacteriological tests.

At the end of the study, the following end points are measured: mammary secretion pre calving, conception rate by pulse; fertility rate; fertility after calving and/or weaning; cow weight after calving and/or weaning; infection diseases in cow.

Efficacy of cessation of milk secretion following administration of casein-derived peptides is measured as follows: high success: cessation within 1-4 days; success 5-8 days; low success 9-15 days; unsuccessful 16 or more days for cessation of milk secretion.

The PPI length is measured from calving and/or weaning day up to ovulation day. Successful outcome is evaluated as follows: high success—less than 12 to 20 days; success—21 to 24 days; low success—25 to 29 days; unsuccessful—30 or more days.

Insemination rate is evaluated as follows: high success—80 or more percentage; success—65 to 79 percentages; low success—50 to 64 percentages; unsuccessful—49 or less percentages.

The clinical trial is stopped at any of the following conditions: completion of study; screening failure; refusal by herd's owner; the cow suffers intolerable adverse events; the calf suffers intolerable adverse events; violation of inclusion/exclusion criteria; any factor or condition that would in the opinion of the investigator necessitate withdrawal from the study.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method for estrus induction in a lactating livestock animal comprising administering to the animal an effective amount of at least one peptide derived from casein.

2. The method according to claim 1, wherein the estrus occurs from about 2 days to about 120 days after the peptide administration.

3. The method according to claim 2, wherein the estrus occurs from about 2 days to about 50 days after the peptide administration.

4. The method according to claim 1, wherein the at least one peptide is administered in a time point selected from the group consisting of from about 70 days before parturition to about 150 days after parturition; from immediately after parturition to about 90 days after parturition; from immediately after parturition to about 60 days after parturition; and from immediately after parturition to about 30 days after parturition

5. (canceled)

6. (canceled)

7. (canceled)

8. The method according to claim 1, wherein the livestock animal is selected from the group consisting of a cow, a goat, a sheep, a swine, a camel and a buffalo.

9. The method according to claim 1, wherein the at least one casein-derived peptide is applied to a plurality of lactating livestock animals in a herd, thereby synchronizing the estrus of the lactating animals in the herd.

10. The method according to claim 9, wherein the herd is selected from the group consisting of herd of beef cattle, herd of dairy cattle, and herd of swine.

11. The method according to claim 9, further comprising inseminating the plurality of livestock animals in the herd.

12. The method according to claim 11, wherein conception rate after insemination is at least 70% or at least 75%.

13. (canceled)

14. A method for inducing a conception rate of at least 70% in a herd, comprising administering to a plurality of livestock animals in the herd an effective amount of at least one peptide derived from casein.

15. The method according to claim 14, wherein the conception rate is at least 70%.

16. The method according to claim 1, wherein the at least one peptide is a phosphopeptide.

17. (canceled)

18. The method according to claim 16, wherein the phosphopeptide comprises an amino acid sequence as set forth in SEQ ID NO:1, a fragment, an analog or a derivative thereon.

19. The method of claim 18, wherein the phosphopeptide is selected from the group consisting of a phosphopeptide derived from β-casein comprising an amino acid sequences as set forth in SEQ ID NO:2; a phosphor peptide derived from αS1-casein comprising an amino acid sequences as set forth in SEQ ID NO:3; a phosphopeptide derived from αS2-casein comprising an amino acid sequences as set forth in SEQ ID NO:4; a phosphopeptide derived from αS2-casein comprising an amino acid sequences as set forth in SEQ ID NO:5; and a fragment, an analog or a derivative thereof.

20. (canceled)

21. (canceled)

22. The method according to claim 1, wherein the at least one peptide derived from casein is selected from the group consisting of a peptide obtained by hydrolysis of casein and a synthetic peptide.

23. (canceled)

24. The method according to claim 1, wherein the at least one peptide is administered into at least one mammary gland of the lactating animal.

25. The method according to claim 24, wherein the at least one peptide is administered simultaneously to all the mammary glands of the lactating animal.

26. The method according to claim 24, wherein the administration is performed by a method selected from the group consisting of intracanal injection, application via patch attached to the livestock udder and application via soaking of the livestock udder.

27. The method according to claim 1, wherein the at least one peptide is administered at least once.

28. The method according to claim 1, wherein the at least one peptide is administered between one to five times at intervals of from about 6 hours to about 24 hours.

29. A method for inducing stress-free weaning of a suckling offspring comprising administering to a lactating animal at least one day prior to weaning an effective amount of at least one peptide derived from casein.

30. The method according to claim 29, wherein the lactating animal is selected from the group consisting of a cow, a goat, a sheep, a swine, a camel and a buffalo.

31. The method according to claim 29 wherein weaning is commenced in a time point selected from the group consisting of from about 2 weeks to about 15 weeks after parturition and from about 4 weeks to about 8 weeks after parturition.

32. (canceled)

33. The method according to claim 29, wherein the offspring and the lactating animal do not essentially lose weight after weaning.

34. The method according to claim 29, wherein the body condition score of the lactating animal and the offspring is essentially the same after weaning as before weaning.

35. The method according to claim 29, wherein the at least one peptide is a phosphopeptide.

36. (canceled)

37. The method according to claim 35, wherein the phosphopeptide comprises an amino acid sequence as set forth in SEQ ID NO:1, a fragment, an analog or a derivative thereof.

38. The method of claim 37, wherein the phosphopeptide is selected from the group consisting of a phosphopeptide derived from β-casein comprising an amino acid sequences as set forth in SEQ ID NO:2; a phosphopeptide derived from αS1-casein comprising an amino acid sequences as set forth in SEQ ID NO:3; a phosphopeptide derived from αS2-casein comprising an amino acid sequences as set forth in SEQ ID NO:4; a phosphopeptide derived from αS2-casein comprising an amino acid sequences as set forth in SEQ ID NO:5; a fragment, an analog or a derivative thereof.

39. (canceled)

40. (canceled)

41. The method according to claim 29, wherein the at least one peptide derived from casein is selected from the group consisting of a peptide obtained by hydrolysis of casein and a synthetic peptide.

42. (canceled)

43. The method according to claim 29, wherein the at least one peptide is administered into at least one mammary gland of the lactating animal.

44. The method according to claim 43, wherein the at least one peptide is administered simultaneously to all the mammary glands of the lactating animal.

45. The method according to claim 43, wherein the administration is performed by a method selected from the group consisting of intracanal injection, application via patch attached to the livestock udder and application via soaking of the livestock udder.

46. The method according to claim 29, wherein the at least one peptide is administered at a time point selected from the group consisting of between about 1 days to about 10 days prior to weaning and about 3 days prior to weaning.

47. (canceled)

48. The method according to claim 29 wherein the at least one peptide is administered between one to ten times at intervals of from about 6 hours to about 24 hours.

49. The method according to claim 29, wherein the comfort of the lactating animal and the offspring is not negatively affected by the induced weaning.

50. The method according to claim 14, wherein the at least one peptide is a phosphopeptide.

51. The method according to claim 14, wherein the at least one peptide derived from casein is selected from the group consisting of a peptide obtained by hydrolysis of casein and a synthetic peptide.

52. The method according to claim 14, wherein the at least one peptide is administered into at least one mammary gland of the lactating animal.

53. The method according to claim 14, wherein the at least one peptide is administered at least once.

54. The method according to claim 14, wherein the at least one peptide is administered between one to five times at intervals of from about 6 hours to about 24 hours.
